(19) United States
(12) Patent Application Publication
(10) Pub. No.: US 2007/0275396 A1
(43) Pub. Date: Nov. 29, 2007

(54) Detection of DNA hybridization with a carbon nanotube label

(76) Inventor: Ming Zheng, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/716,160

(22) Filed: Mar. 9, 2007

Related U.S. Application Data
(60) Provisional application No. 60/783,639, filed on Mar. 17, 2006.

(51) Int. Cl. C12Q 1/68

(52) U.S. Cl. 435/6

(57) ABSTRACT

Methods are provided for the detection and identification of target nucleic acids using a system comprising a nanotube—nucleic acid complex. The complex is comprised of a singly dispersed carbon nanotube and a dispersant nucleic acid molecules that is associated with the carbon nanotube in a non-covalent fashion. Portions of the target nucleic acid and the dispersant nucleic acid associated with the nanotube are complementary to each other allowing for hybridization when the two come in contact under the appropriate conditions. The hybridization event is reported through changes in the electrochemical, conductive or spectral properties of the nanotube component.

This application claims the benefit of U.S. Provisional Application 60/783,639, filed Mar. 17, 2006.

### FIELD OF INVENTION

The invention relates to the hybridization and detection of DNA using carbon nanotubes. More specifically, carbon nanotubes (CNT) associated with DNA in a non-covalent fashion are used as reporters to detect hybridization of target nucleic acids.

### BACKGROUND

There is an increasing need for rapid, small scale and highly sensitive detection of biological molecules in medical, bioterrorism, food safety, and research applications. Nanostructures such as carbon nanotubes display physical and electronic properties amenable to use in miniature devices. Carbon nanotubes (CNT's) are rolled up graphene sheets having a diameter on the nanometer scale and typical lengths of up to several micrometers.

Measurement of DNA hybridization has been accomplished using electrochemical detection. Hybridization of a single-stranded polyC DNA probe attached to carbon nanotubes with the complementary polyG DNA strand was detected amperometrically. (J. Li et al. (2003) Nano Lett. 3:597). U.S. Patent Appn. 2005/0019791 describes a method to detect DNA hybridization by measuring electric or electrochemical changes on a biochip comprising a bio-receptor attached by means of a functional group on the surface of a laminated CNT film or pattern on a substrate modified with amine groups.

Spectroscopic methods of detection have the potential to be faster and more sensitive than existing methods. U.S. Patent Appn. 2005/0164211 describes the use of derivatized carbon nanotubes as oligonucleotide probes labels in sequencing of nucleic acids. The nanotube labels are covalently attached to oligonucleotides and are distinguished by emission spectra upon excitation by an electron beam or UV laser.

In U.S. Patent No. 20040132072 it was disclosed that stabilized solutions of nucleic acid molecules have the ability to disperse and solubilize carbon nanotubes, resulting in the formation of nanotube-nucleic acid complexes.

Star et al (PNAS, Vol 103, No. 4, p. 921 (2006)) disclose the detection of DNA hybridization between single stranded DNA associated with carbon nanotube field-effect transistors and target DNA. Similarly Heller et al, (Science, Vol 311, No. 5760, p. 508 (2006)) disclose the use of DNA wrapped carbon nanotubes as reporters for hybridization events based on the modulation of the dielectric environment of the CNT in response to hybridization.

A need exists for the facile and sensitive detection of hybridization as a method for the identification of target nucleic acid molecules in diagnostic regimes. Applicants have met the stated need through the development of a system comprising a DNA wrapped CNT having a region of complimentarity to a target nucleic acid to be identified. Hybridization of the target nucleic acid to the wrapping DNA alters the properties of the CNT, allowing for detection of the event.

### SUMMARY OF THE INVENTION

The invention relates to the detection and identification of target nucleic acids using a system comprising a nanotube—nucleic acid complex. The complex is comprised of a singly dispersed carbon nanotubes and a dispersant nucleic acid molecules that are associated with the carbon nanotube in a non-covalent fashion. Portions of the target nucleic acid and the dispersant nucleic acid associated with the nanotube are complementary to each other allowing for hybridization when the two come in contact under the appropriate conditions. The hybridization event is reported through changes in the electrochemical, conductive or spectral properties of the nanotube component.

Accordingly the invention provides a method of labeling a target nucleic acid molecule, comprising:
a) providing a target nucleic acid molecule having a target sequence;
b) providing a solution containing a population of singly dispersed, carbon nanotube—nucleic acid complexes, each complex comprising a single walled carbon nanotube non-covalently associated with a dispersant nucleic acid molecule wherein the nucleic acid molecule comprises a sequence complementary to the target sequence;
c) hybridizing the target nucleic acid molecule of step (a) to the dispersant nucleic acid molecule of step (b) to form a hybridized complex wherein the target nucleic acid molecule is labeled; and
d) optionally recovering the labeled target nucleic acid molecule.

In another embodiment the invention provides a method for the detection of a target nucleic acid molecule, comprising:
a) providing a target nucleic acid molecule having a target sequence;
b) providing a solution containing a population of singly dispersed, carbon nanotube—nucleic acid complexes, each complex comprising a single walled carbon nanotube non-covalently associated with a dispersant nucleic acid molecule wherein the nucleic acid molecule comprises a sequence complementary to the target sequence;
c) hybridizing the target nucleic acid molecule of step (a) to the dispersant nucleic acid molecule of step (b) to form a hybridized complex; and
d) detecting the hybridization of step (c) by measuring change in properties of the carbon nanotube, before and after hybridization, wherein the target nucleic acid molecule is detected.
   In a similar embodiment the invention provides a method for the detection of a target nucleic acid molecule, comprising:
a) providing a target nucleic acid molecule having a target sequence;
b) providing a solution containing a population of singly dispersed, carbon nanotube—nucleic acid complexes, each complex comprising a single walled carbon nanotube non-covalently associated with a dispersant nucleic acid molecule wherein the nucleic acid molecule comprises a sequence complementary to the target sequence;
c) proving a solid support comprising a linking nucleic acid molecule, further comprising a first hybridization sequence complementary to the target sequence and a second hybridization sequence complementary to at least a portion of the dispersant nucleic acid molecule:
d) hybridizing the target nucleic acid molecule of step (a) and the dispersant nucleic acid molecule of step (b) to the linking nucleic acid molecule of step (c) to form an immobilized complex; and
e) detecting the hybridization of step (d) by measuring change in properties of the carbon nanotube, before and after hybridization wherein the target nucleic acid is detected.
   In a related embodiment the invention provides a method for the detection of a target nucleic acid molecule, comprising:
a) providing a target nucleic acid molecule having a target sequence wherein the target sequence is immobilized on a solid support;
b) providing a solution containing a population of singly dispersed, carbon nanotube—nucleic acid complexes, each complex comprising a single walled carbon nanotube non-covalently associated with a dispersant nucleic acid molecule wherein the nucleic acid molecule comprises a sequence complementary to the target sequence;
c) hybridizing the target nucleic acid molecule of step (a) and the dispersant nucleic acid molecule of step (b) to form an immobilized complex; and
d) detecting the hybridization of step (c) by measuring change in properties of the carbon nanotube, before and after hybridization, wherein the target nucleic acid is detected.

### BRIEF DESCRIPTION OF THE FIGURES

FIG. 1 illustrates a SWNT comprising a non-covalently associated single stranded nucleic acid having a loop structure and a terminal portion free for hybridization.

FIG. 2 A illustrates DNA hybridization detection using magnetic beads labeled with a target sequence complementary to a portion of the dispersant nucleic acid sequence. FIG. 2B illustrates DNA hybridization detection using magnetic beads labeled with a target sequence complementary to a linking sequence which in turn has complementarity to the dispersant nucleic acid associated with the CNT

FIG. 3 shows the results of the agglomeration from the successful hybridization in Example 3.

FIG. 4 is a graph of UV-Vis absorption spectroscopy measurements from Example 4 demonstrating selective hybridization using DNA wrapped CNTs.

### DETAILED DESCRIPTION OF THE INVENTION

The invention provides methods of the detection of a nucleic acid hybridization event where the event is reported by a CNT label. The method employs a nanotube—nucleic acid complex formed between and SWNT and a dispersant nucleic acid molecule. The dispersant nucleic acid has a region of complementarity to a target sequence located within at target nucleic acid to be detected. Hybridization between the target and the dispersant nucleic acid results in changes in the properties of the associated CNT that may be detected by conductive or spectral detection means.

The invention may be used to detect nucleic acids associated with disease states or diagnostic of pathogens in the medical, biomedical and food safety industries. Additionally, the methods of the invention have application in any area where detection of specific nucleic acids are needed.

The following abbreviations and definitions may be used for the interpretation of the specification and the claims.

“cDNA” means complementary DNA

“PNA” means peptide nucleic acid

“ssDNA” means single stranded DNA

“CNT” means carbon nanotube

“MWNT” means multi-walled nanotube

“SWNT” means single walled nanotube

As used herein, a “nucleic acid molecule” is a polymer of RNA, DNA or a peptide nucleic acid (PNA that is single- or double-stranded, optionally containing synthetic, non-natural or altered nucleotide bases. A nucleic acid molecule in the form of a polymer of DNA may be comprised of one or more segments of cDNA, genomic DNA or synthetic DNA.
“Target nucleic acid molecules” are those that are intended for detection or identification by the present methods. Target nucleic acid molecules will typically comprise portions or domains that are diagnostic of that sequence and to which “regions of complementarity” may be designed.

The term “peptide nucleic acids” refers to a material having stretches of nucleic acid polymers linked together by peptide linkers.

The term “complementary” or “complementarity” as used herein, include the natural binding of polynucleotides under permissive salt and temperature conditions by base-pairing. For example, the sequence “A-G-T” binds to the complementary sequence “T-C-A.” Complementarity between two single-stranded molecules may be “partial,” in which only some of the nucleic acid binds, or it may be complete when total complementarity exists between the single-stranded molecules. The degree of complementarity between nucleic acid strands has significant effects on the efficiency and strength of hybridization between nucleic acid strands.

The term “region of complementarity” refers to a specific portion of a nucleic acid molecule that is designed to hybridize to a complementary strand of another nucleic acid molecule.

The term “hybridization” refers to any process by which a nucleic acid sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferable a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “carbon nanotube” refers to a hollow article composed primarily of carbon atoms. The carbon nanotube can be doped with other elements, e.g., metals. The nanotubes typically have a narrow dimension (diameter) of about 1-200 nm and a long dimension (length), where the ratio of the long dimension to the narrow dimension, i.e., the aspect ratio, is at least 5. In general, the aspect ratio is between 10 and 2000.

A “dispersant nucleic acid molecule” is a nucleic acid that is non-covalently associated with a CNT. Typically dispersant nucleic acid molecules are single stranded and effectually disperse a population of CNT's in a solution. Dispersant nucleic acid molecules will generally comprise a region of complementarity with a target sequence for the hybridization and identification of a target nucleic acid. By “non-covalently associated” it is meat that the dispersant nucleic acids are associated with the CNT in a manner that does not involve covalent bonding, i.e via hydrogen bonding or Van der Walls forces for example. CNT that comprise a dispersant nucleic acid molecule are refereed to herein as “nanotube-nucleic acid complexes”.

By “detection” it is meant the indication of the presence and/or quantity of the target nucleic acid. Detection of the target nucleic acid as described herein is accomplished indirectly via measuring the change in properties of the CNT in the nanotube-nucleic acid complex. Detection therefore will consist of measuring the conductive, spectral or electrical properties of the CNT in the complex.

The letters “A”, “G”, “T”, “C” when referred to in the context of nucleic acids will mean the purine bases adenine (C₅H₅N₅) and guanine (C₅H₅N₅O) and the pyrimidine bases thymine (C₅H₆N₂O₂) and cytosine (C₄H₅N₃O), respectively.

As used herein the term “stabilized solution of nucleic acid molecules” refers to a solution of nucleic acid molecules that are solubilized and in a relaxed secondary conformation.

The term “binding-pair” includes any of the class of immune-type binding-pairs, such as, antigen/antibody, antigen/antibody fragment, or hapten/anti-hapten systems; and also any of the class of nonimmune-type binding-pairs, such as biotin/avidin, biotin/streptavidin, folic acid/folate binding protein, hormone/hormone receptor, lectin/specific carbohydrate, enzyme/enzyme enzyme/substrate, enzyme/inhibitor, or, vitamin B12/intrinsic factor. They also include complementary nucleic acid fragments (including DNA sequences, RNA sequences, and peptide nucleic acid sequences), as well as Protein A/antibody or Protein G/antibody, and polynucleotide/polynucleotide binding protein. Binding pairs may also include members that form covalent bonds, such as, sulfhydryl reactive groups including maleimides and haloacetyl derivatives, and amine reactive groups such as isothiocyanates, succinimidyl esters, carbodiimides, and sulfonyl halides.

The term “agitation means” refers to a devices that facilitate the dispersion of nanotubes and nucleic acids. A typical agitation means is sonication.

The term “denaturant” as used herein refers to substances effective in the denaturation of DNA and other nucleic acid molecules.

The term “solid support” means a material suitable for the immobilization of a nanotube-nucleic acid complex. Typically the solid support provides an attachment for a target sequence. One preferred example of a solid support are magnetic beads.

The terms “protein”, “peptide”, “polypeptide” and “oligopeptide” are herein used interchangeably to refer to two or more covalently linked, naturally occurring or synthetically manufactured amino acids.

The term “label” refers to any atom or molecule that can be attached or associated with a nucleic acid. In the context of the present invention CNT's are used as labels to report a hybridization event. As such, these CNT labels are also “reporters”.

The term “reporter” refers to any atom or molecule that is used as a “label” to provide a detectable (preferably quantifiable) signal, and which can be attached to a nucleic acid.

The term “oligonucleotide”, refers to polydeoxyribonucleotides (containing 2-deoxy-D-ribose), to polyribonucleotides (containing D-ribose) and to any polynucleotide, which is a ribo sugar-phosphate backbone consisting of an N-glycoside of a purine or pyrimidine base, or modified purine or pyrimidine base. There is no intended distinction between the length of a “nucleic acid”, “polynucleotide” or an “oligonucleotide”.

Standard recombinant DNA and molecular biology techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

One aspect of the invention is directed to a method of labeling or identifying a target nucleic acid molecule. The target nucleic acid comprises a target sequence that is diagnostic of the target molecule. The target nucleic acid is contacted with a nanotube—nucleic acid complex comprising a dispersant nucleic acid non-covalently associated with a CNT. The dispersant nucleic acid comprises a region of complementarty to the target sequence. When the target nucleic acid comes in contact with the nanotube—nucleic acid complex a hybridization event occurs between the target sequence and the region of complementarity on the dispersant nucleic acid. The hybridization event is reported by measuring the changes in the CNT before and after the hybridization event.

Target Nucleic Acid Molecules.

Target nucleic acid molecules may be from any source, however must comprise a region (target sequence) that may be use to uniquely identify the target. Typically targets will be from a variety of biological sources inducing but not limited to skin, plasma, serum, spinal fluid, lymph fluid, synovial fluid, urine, tears, blood cells, organs, and tumors. Additionally targets may originate from bacteria, viruses, yeast, fungi, algae, and the cells of higher plants and mammals. Alternatively, targets may be from synthetic sources.

In many cases the target may be indicative of a particular disease state. Disease states that are readily identified on the basis of nucleic acid detection include but are not limited to congenital diseases such as adrenal hyperplasia (involving enzymes required for synthesis of cortisol, aldosterone, and sex steroids in the adrenal gland), myotonic dystrophy, hypothyroidism, cataract development, tay-sachs disease; as well as other diseases having well described genetic markers such as retinoblastoma, blood group antigen mutations, neurofibromatosis and most cancers including breast cancer.

The target nucleic acid molecules may be of any size or length, be of any base composition and take any conformation provided that at least a portion of the sequence permits hybridization. Target nucleic acids may be single stranded, double stranded or comprise peptide portions as with peptide nucleic acids (PNA).

Carbon Nanotube—Nucleic Acid Complex

An integral part of the present invention is the nanotube—nucleic acid complex which is comprised of a CNT associated with a dispersant nucleic acid.

Carbon nanotubes of the invention are generally about 0.5-2 nm in diameter where the ratio of the length dimension to the narrow dimension, i.e., the aspect ratio, is at least 5. In general, the aspect ratio is between 10 and 2000. Carbon nanotubes are comprised primarily of carbon atoms, however may be doped with other elements, e.g., metals. The carbon-based nanotubes of the invention can be either multi-walled nanotubes (MWNTs) or single-walled nanotubes (SWNTs). A MWNT, for example, includes several concentric nanotubes each having a different diameter. Thus, the smallest diameter tube is encapsulated by a larger diameter tube, which in turn, is encapsulated by another larger diameter nanotube. A SWNT, on the other hand, includes only one nanotube.

Carbon nanotubes (CNT) may be produced by a variety of methods, and are additionally commercially available. Methods of CNT synthesis include laser vaporization of graphite (A. Thess et al. Science 273, 483 (1996)), arc discharge (C. Journet et al., Nature 388, 756 (1997)) and HiPC® (high pressure carbon monoxide) process (P. Nikolaev et al. Chem. Phys. Lett. 313, 91-97 (1999)). Chemical vapor deposition (CVD) can also be used in producing carbon nanotubes (J. Kong et al. Chem. Phys. Lett. 292, 567-574 (1998); J. Kong et al. Nature 395, 878-879 (1998); A. Cassell et al. J. Phys. Chem. 103, 6484-6492 (1999); H. Dai et al. J. Phys. Chem. 103, 11246-11255 (1999)).

Additionally CNT's may be grown via catalytic processes both in solution and on solid substrates (Yan Li, et al., Chem. Mater.; 2001; 13(3); 1008-1014); (N. Franklin and H. Dai Adv. Mater. 12, 890 (2000); A. Cassell et al. J. Am. Chem. Soc. 121, 7975-7976 (1999)).

The carbon nanotube—nucleic acid complex can be prepared as described in U.S. Appn. No. 20040132072, herein incorporated entirely by reference. The complex is prepared generally by dispersing a population of bundled carbon nanotubes by contacting the bundled nanotubes with a stabilized solution of dispersant nucleic acid molecules, forming nanotube-nucleic acid complexes based on non-covalent interactions between the nanotube and the nucleic acid molecule.

Dispersant nucleic acid molecules of the invention may be of any type and from any suitable source and include but are not limited to DNA, RNA and peptide nucleic acids. The nucleic acid molecules may be either single stranded or double stranded and may optionally be functionalized at any point with a variety of reactive groups, ligands or agents. The nucleic acid molecules of the invention may be generated by synthetic means or may be isolated from nature by protocols well known in the art (Sambrook supra).

Peptide nucleic acids (PNA) are particularly useful in the present invention as they possess the double functionality of both nucleic acids and peptides. Methods for the synthesis and use of PNA's are well known in the art, see for example Antsypovitch, S. I. Peptide nucleic acids: structure Russian Chemical Reviews (2002), 71(1), 71-83.

The dispersant nucleic acid molecules may have any composition of bases and may even consists of stretches of the same base (poly A or polyT for example) without impairing the ability of the nucleic acid molecule to disperse the bundled nanotube. Preferably the nucleic acid molecules will be less than about 2000 bases where less than 1000 bases is preferred and where from about 5 bases to about 1000 bases is most preferred. Generally the ability of nucleic acids to disperse carbon nanotubes appears to be independent of sequence or base composition, however there is some evidence to suggest that the less G-C and T-A base-pairing interactions in a sequence, the higher the dispersion efficiency, and that RNA and varieties thereof is particularly effective in dispersion and is thus preferred herein. Nucleic acid molecules suitable for use in the present invention include but are not limited to those having the general formula:
1. An wherein n=1-2000;
2. Tn wherein n=1-2000;
3. Cn wherein n=1-2000;
4. Gn wherein n=1-2000;
5. Rn wherein n=1-2000, and wherein R may be either A or G;
6. Yn wherein n=1-2000, and wherein Y may be either C or
7. Mn wherein n=1-2000, and wherein M may be either A or C.
8. Kn wherein n=1-2000, and wherein K may be either G or T;
9. Sn wherein n=1-2000, and wherein S may be either C or
10. Wn wherein n=1-2000, and wherein W may be either A or T;
11. Hn wherein n=1-2000, and wherein H may be either A or C or T;
12. Bn wherein n=1-2000, and wherein B may be either C or G or T;
13. Vn wherein n=1-2000, and wherein V may be either A or C or G;
14. Dn wherein n=1-2000, and wherein D may be either A or G or T; and
15. Nn wherein n=1-2000, and wherein N may be either A or C or T or G;

In addition the combinations listed above the person of skill in the art will recognize that any of these sequences may have one or more deoxyribonucleotides replaced by ribonucleotides (i.e., RNA or RNA/DNA hybrid) or one or more sugar-phosphate linkages replaced by peptide bonds (i.e. PNA or PNA/RNA/DNA hybrid).

One important aspect of the dispersant nucleic acid molecules is that it be engineered or designed to incorporate a region of complementarity to that of a target sequence contained with a target nucleic acid for detection.

Once the nucleic acid molecule has been prepared it may be stabilized in a suitable solution. It is preferred if the nucleic acid molecules are in a relaxed secondary conformation and only loosely associated with each other to allow for the greatest contact by individual strands with the carbon nanotubes. Stabilized solutions of nucleic acids are common and well known in the art (see Sambrook supra) and typically include salts and buffers such as sodium and potassium salts, and TRIS (Tris(2-aminoethyl)amine), HEPES (N-(2-hydroxyethyl)piperazine-N′-(2-ethanesulfonic acid), and MES (2-(N-Morpholino)ethanesulfonic acid. Preferred solvents for stabilized nucleic acid solutions are those that are water miscible where water is most preferred.

Once the nucleic acid molecules are stabilized in a suitable solution they may be contacted with a population of bundled carbon nanotubes. It is preferred, although not necessary if the contacting is done in the presence of an agitation means of some sort. Typically the agitation means employs sonication for example, however may also include, devices that produce high shear mixing of the nucleic acids and nanotubes (i.e. homogenization), or any combination thereof. Upon agitation the carbon nanotubes will become dispersed and will form nanotube-nucleic acid complexes comprising at least one nucleic acid molecule loosely associated with the carbon nanotube by hydrogen bonding or some non-covalent means.

The process of agitation and dispersion may be improved with the optional addition of nucleic acid denaturing substances to the solution. Common denaturants include but are not limited to formamide, urea and guanidine. A non-limiting list of suitable denaturants may be found in Sambrook supra.

Additionally temperature during the contacting process will have an effect on the efficacy of the dispersion. Agitation at room temperature or higher was seen to give longer dispersion times whereas agitation at temperatures below room temperature (23° C.) were seen to give more rapid dispersion times where temperatures of about 4° C. are preferred.

Once the nanotube-nucleic acid molecule complexes are formed they can optionally be separated from solution. Where the nucleic acid has been functionalized by the addition of a binding pair for example separation could be accomplished by means of immobilization thought the binding pair as discussed below. However, where the nucleic acid has not been functionalized an alternate means for separation must be found. Applicants have provided a novel separation method involving either gel electrophoresis chromatography or a phase separation method that is rapid and facile and permits the separation of nanotube-nucleic acid complexes into discreet fractions based on size or charge. These methods have been applied to the separation and recovery of coated nanoparticles (as described in U.S. Ser. No. 10/622,889 incorporated herein by reference) and have been found useful here.

Gel electrophoresis is a commonly used method in biochemistry and molecular biology to separate macromolecules such as proteins and nucleic acids. The gel serves as a sieving medium to separate the macromolecules on the basis of size. In the present invention, the gel can be made from agarose or polyacrylamide. Methods for preparing suitable gels are well known and exemplified in Sambrook, supra, particularly Chapter 6 (entirely incorporated herein by reference). Suitable agarose gels have an agarose concentration between 0.6 and 6% (weight per volume), while suitable polyacrylamide gels have an acrylamide concentration between 3.5 and 20% (weight per volume). It is well know in the art that the concentration of the gel to be used depends on the size of the molecules being separated. Specifically, higher gel concentrations provide better separation for smaller molecules, while lower gel concentrations are used to separate larger molecules. The gel concentration to be used for a given nanoparticle fractionation can be determined by routine experimentation. The preferred gel of the present invention is a 1% or lower agarose gel.

In order to determine the average particle size of the complexes a densifying agent may be added to an aqueous solution of the complexes. The purpose of densifying agent is to increase the specific gravity of the nanoparticle solution to facilitate loading of the solution into the gel. Suitable densifying agents are well known and include, but are not limited to, glycerol, sucrose, and Ficoll (a nonionic, synthetic polymer of sucrose, approximate molecular weight of 400,000, available from Sigma, St. Louis, Mo.). The complex solution is then added to the wells in the gel. The complexes migrate according to their apparent molecular weight and size of any particular complex may be determined by using molecular weight standards.

Alternatively the complexes may be separated by two phase separation methods. In this method nanotube-nucleic acid complexes in solution are fractionated by adding a substantially water-miscible organic solvent in the presence of an electrolyte. The amount of the substantially water-miscible organic solvent added depends on the average particle size desired. The appropriate amount can be determined by routine experimentation. Typically, the substantially water-miscible organic solvent is added to give a concentration of about 5% to 10% by volume to precipitate out the largest particles. The complexes are collected by centrifugation or filtration. Centrifugation is typically done using a centrifuge, such as a Sorvall® RT7 PLUS centrifuge available from Kendro Laboratory Products (Newtown, Conn.), for about 1 min at about 4,000 rpm. For filtration, a porous membrane with a pore size small enough to collect the complex size of interest can be used. Optionally, sequential additions of the substantially water-miscible organic solvent are made to the complex solution to increase the solvent content of the solution and therefore, precipitate out complexes of smaller sizes.

Target—Dispersant Hybridization

The invention relies on the hybridization between the target nucleic acid molecule and the dispersant nucleic acid that is associated with the CNT in the nanotube—CNT complex.

Effective nucleic acid hybridization protocols are common and well known in the art. (Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1) Typically the target and complex comprising the dispersant nucleic acid must be mixed under conditions which will permit nucleic acid hybridization. This involves contacting the nucleic acids in the presence of an inorganic or organic salt under the proper concentration and temperature conditions. The nucleic acids must be in contact for a long enough time that any possible hybridization between the probe and sample nucleic acid may occur. The concentration of dispersant nucleic acid or target in the mixture will determine the time necessary for hybridization to occur. The higher the nucleic acid concentration the shorter the hybridization incubation time needed. Optionally a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, Nucl. Acids Res. 19:5143-5151 (1991)). Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1M sodium chloride, about 0.05 to 0.1M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia Inc.) (about 300-500 kilodaltons), polyvinylpyrrolidone (about 250-500 kdal), and serum albumin. Also included in the typical hybridization solution will be unlabeled carrier nucleic acids from about 0.1 to 5 mg/mL, fragmented nucleic DNA, e.g., calf thymus or salmon sperm DNA, or yeast RNA, and optionally from about 0.5 to 2% wt./vol. glycine. Other additives may also be included, such as volume exclusion agents which include a variety of polar water-soluble or swellable agents, such as polyethylene glycol, anionic polymers such as polyacrylate or polymethylacrylate, and anionic saccharidic polymers, such as dextran sulfate.

Detection of Hybridization Event

The incorporation of the CNT into the present method allows for sensitive detection of the hybridization event based on the ability to measure subtle differences in the properties of the CNT before and after the hybridization. For example, small changes in the electrochemical, conductive and spectral properties of the CNT may be correlated with hybridization.

It is well known for example that CNT's as part of field-effect sensors will demonstrate measurable changes in surface charge in response to changes at the surface of the CNT i.e hybridization (Star et al., supra). Similarly CNT's have been used as electrodes where changes in electrical conductivity has been correlated with surface changes (Cai et al., Anal. Bioannal. Chem. 375, 287, (2003)). Alternatively it has been shown that the native fluorescence of SWNT will undergo a red shift in response to changes in the charge of an associated nucleic acid (Heller et al., supra). Although not meant to be limiting, any of the above methods may be used as a detection means to report on the hybridization event.

Assay Formats

Fundamental to the methods of the invention is the hybridization of the target nucleic acid to the dispersant nucleic acid and the reporting of the hybridization event through by monitoring changes in the electrochemical, conductive, or spectral properties of the associated CNT. The skilled person will recognize that the methods of the invention may be employed in a multiplicity of formats.

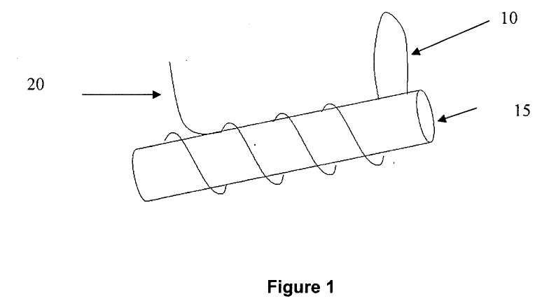

For example, the system may be used in a homogeneous format where neither the target nucleic acid nor the nanotube-nucleic acid complex is immobilized. In this format the nanotube-nucleic acid complex is contacted in the appropriate environment with a sample suspected of containing the target nucleic acid under conditions that will facilitate hybridization. The dispersant nucleic acid is associated with the CNT in a non-covalent manner and in a way that makes regions of the nucleic cid available for hybridization. Referring to FIG. 1 for example, the dispersant nucleic acid is wrapped about the CNT (15) in a manner that allows for loop structures (10) or free ends (20) of the nucleic acids to be exposed and available for hybridization with the target sequence. The properties of the CNT portion of the complex may be monitored before and after hybridization as an indicator of the hybridization event. For example, prior to contacting the complex with the target a fluorescence spectrum may be obtained from the complex. The spectrum is obtained again after the complex is contacted with the target and those complexes showing a spectral shift will be reporting a hybridization event and the identification of the target in the sample.

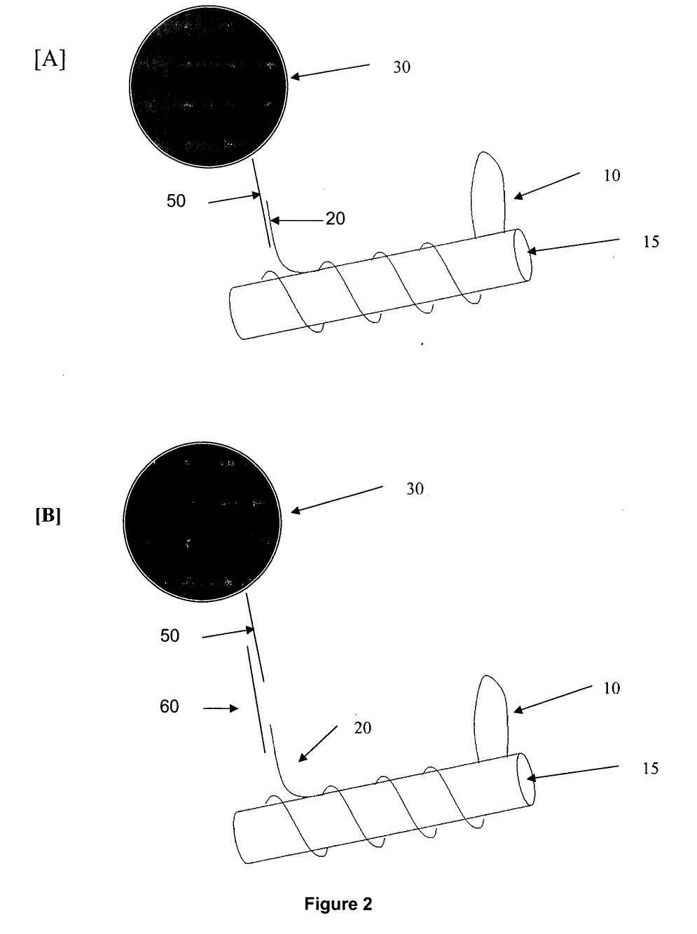

Alternatively the methods of the invention may proceed in heterogeneous fashion. For example the nanotube—nucleic acid complex may form an electrically conducting layer as part of an FET device where changes in conductivity may be measured. Contacting the target with the layer of nanotube—nucleic acid complexes under hybridizing conditions will give rise to the hybridization event which can then be detected via a change in the conductive properties of the FET device. In this format the target sequence in the target nucleic acid may be complementary to a portion of the dispersant nucleic acid directly. Alternatively the target could be recognized through a linking sequence that links the target and the dispersant nucleic acid. Another embodiment of this concept is illustrated in FIG. 2.

Referring to FIG. 2A, the target nucleic acid (50) may be immobilized on a solid support. Methods of immobilization of nucleic acids to surfaces is common and well known in the art, as discussed for example in Aboytes et al, Beginner's Guide to Microarrays (2003), 141. Editor(s): Blalock, Eric M. Publisher: Kluwer Academic Publishers, Norwell, Mass; Urbina et al, Innovation and Perspectives in Solid Phase Synthesis & Combinatorial Libraries: Peptides, Proteins and Nucleic Acids—Small Molecule Organic Chemistry Diversity, Collected Papers, International Symposium, 6th, York, United Kingdom, Aug. 31-Sep. 4, 1999 (2001), Meeting Date 1999, 37-42. Editor(s): Epton, Roger. Publisher: Mayflower Scientific Ltd., Kingswinford, UK.; Henke et al., Canadian Journal of Analytical Sciences and Spectroscopy (1999), 44(2), 61-70. The solid support shown in FIG. 2A, is in the form of a bead (30) however supports may take a variety of forms and be comprised of a multiplicity of different materials, including, but not limited to glass, synthetic polymer supports, such as polystyrene, polypropylene, polyglycidylmethacrylate, substituted polystyrene (e.g., aminated or carboxylated polystyrene; polyacrylamides; polyamides; polyvinylchlorides, etc.); agarose, nitrocellulose, nylon. These materials may be used as films, microtiter plates, wells, beads, slides, particles, pins, pegs, test tubes, membranes or biosensor chips. Alternatively, the supports could comprise magnetic and non-magnetic particles. Suitable supports and their uses are reviewed by H. Weetall, Immobilized Enzymes, Antigens, Antibodies and Peptides, (1975) Marcell Dekker, Inc., New York.

FIG. 2A illustrates a format with the target nucleic acid (50) is immobilized on the support (30) and directly hybridized to a complementary portion of the dispersant nucleic acid (20). An alternative format is illustrated in FIG. 2B. Here the target nucleic acid (50) is first hybridized with a linking nucleic acid (60), which is in turn hybridized with a complementary portion of the dispersant nucleic acid (20). Use of the linking nucleic acid in this fashion lends greater flexibility to the assay methods of the invention by providing a diversity of regions of complementarity for hybridization.

Alternatively it will be possible to further increase the flexibility of the present methods by additionally functionalizing the target and/or the dispersant nucleic acids with members of binding pairs. So for example either one or both of the nucleic acids could be functionalized with a binding member selected from the binding pairs of the class of immune-type binding-pairs, such as, antigen/antibody, antigen/antibody fragment, or hapten/anti-hapten systems; and also any of the class of nonimmune-type binding-pairs, such as biotin/avidin, biotin/streptavidin, folic acid/folate binding protein, hormone/hormone receptor, lectin/specific carbohydrate, enzyme/enzyme enzyme/substrate, enzyme/inhibitor, or, vitamin B12/intrinsic factor. Binding pairs may also include members that form covalent bonds, such as, sulfhydryl reactive groups including maleimides and haloacetyl derivatives, and amine reactive groups such as isothiocyanates, succinimidyl esters, carbodiimides, and sulfonyl halides. The use of these binding pairs will allow for either the capture of functionalized nucleic acids or other analytes that may be desirable to detect.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Preparation of DNA-Wrapped Carbon Nanotubes

This example describes preparation of DNA-CNT materials to be used for the subsequent EXAMPLES. Unpurified single wall carbon nanotubes from Southwest Nanotechnologies (SWeNT, Norman, Okla.) and single-stranded DNA of defined sequence were used as dispersion agents. Dispersion was done as described in U.S. patent application Ser. No. 10/716,346 herein incorporated by reference. The materials were used as received without further modification. Single-stranded DNA (ssDNA) oligonucleotides were purchased from Integrated DNA Technologies, INC (Coralville, Iowa). In a typical experiment, 10 mg of CNT were suspended in 10 mL of 1×SSC buffer (0.15M NaCl, 0.015M sodium citrate), then sonicated for 2 min. with a TORBEO 130-Watt Ultrasonic Processor (Cole-Parmer Instrument Company, Vernon Hills, Ill.). Nucleic acids were dissolved in H₂O to give a final concentration of 10 mg/mL. 50 μL of the CNT suspension and 5 μL of 10 mg/mL nucleic acid solution were added to 200 μL of H₂O to give a final volume of 255 μL. The mixture was sonicated for 3 min., followed by 90 min of centrifugation at 16,000 g (Biofuge fresco, Kendro Laoratory Products, Newtown, Conn.). The supernatant was then removed for spectroscopic measurement. Absorption spectra from 400 nm to 900 nm were recorded using Ultrospec 3300 UV-Vis spectrophotometer (Amersham Biosciences, Piscataway, N.J.). The 730 nm peak was taken as a measure of the yield of the dispersion process.

Removal of free DNA from the dispersion mixture was done by using size-exclusion chromatography (SEC), as described in U.S. application Ser. No. 11/227,800, herein incorporated by reference. The SEC step is performed to eliminate any free DNA which may compete with the CNT bound DNA for hybridization in the experiments to be described below. A size exclusion column Superdexm™ 200 (16/60, prep grade) from Amersham Biosciences (Piscataway, N.J.)) was chosen for the HPLC purification. A volume of 2 mL of DNA-dispersed carbon nanotubes at a concentration of ˜100 μg/mL was injected into the column mounted on a BioCAD/SPRINT HPLC system (Applied Biosystems, Foster City, Calif.), and eluted by 120 mL of a pH 7 buffer solution containing 40 mM Tris/0.2M NaCl, at a flow rate of 1 mL/min. Fractions were collected in 1 mL aliquots. DNA-CNT hybrids eluted from the column after about 40 mL of elution volume.

Purified DNA-CNTs were then exchanged into pure H₂O using Microcon® centrifugal filter YM-100 (Millipore, Bedford, Mass.) and diluted to a final concentration of about 2 μg/mL. This step served to remove any metallic particles or other impurities that could interfere with device fabrication or function.

The SEC purified DNA-CNTs were typically ˜10 μg/mL in a pH 7.5 buffer composed of 50 mM Tris, 0.5 mM EDTA and 0.2 M NaCl.

### Example 2

### Preparation of DNA-Labeled Magnetic Beads

This example describes preparation of DNA-labeled magnetic beads to be used for the subsequent hybridization experiments. Streptavidin coated magnetic beads at 4 mg/mL were purchased from New England BioLabs (catalog #: S1420G). Biotinylated ssDNAs were purchased from Integrated DNA technologies, Inc. In a typical labeling experiment, 200 μl of the Streptavidin coated magnetic beads were mixed with 10 μl of biotinylated ssDNA (10 mg/mL). After 1 hr incubation at room temperature, the DNA labeled beads were collected by a magnet, and washed with 200 μl of 0.15 M NaCl solution. The washed pellet was then re-suspended in 100 μl of 0.15 M NaCl solution.

### Example 3

### DNA Hybridization

### General Scheme

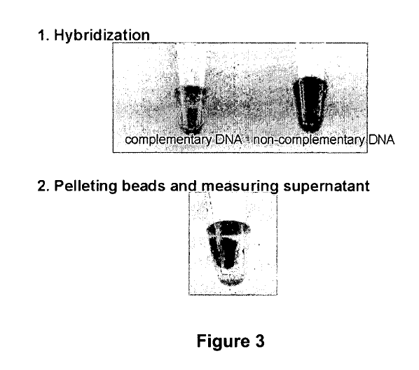

FIGS. 1-3 illustrate the main concept and procedure of DNA hybridization detection using DNA-wrapped carbon nanotubes. A DNA strand wrapped onto a CNT can be viewed as in a dynamic equilibrium between CNT surface bound state and unbound state, as illustrated in FIG. 1. This is especially true for the two end segments of a wrapping strand. The two ends are therefore available for hybridization with its complementary strands labeled on a magnetic bead, as illustrated in FIG. 2. In a typical hybridization experiment, 100 μl of DNA-labeled magnetic beads were mixed with 30 μl of SEC purified DNA-CNT solution as prepared in Example 1. Hybridization was allowed to proceed for 1 hr at room temperature. Since two complementary strands were used to label the magnetic beads and to wrap the CNT, respectively, gradual development of agglomeration of the beads was observed during the hybridization process, as shown in FIG. 3. The agglomeration was absent when non-complementary strands were used. To quantitatively assay for the degree of hybridization, beads were pelleted by a magnet and the supernatants were measured by UV-Vis absorption spectroscopy to determine the amount of CNTs left in solution.

### Example 4

### DNA Hybridization

### A Special Case

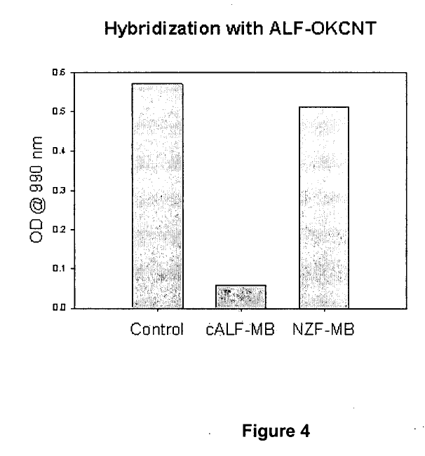

This Example provides a special case for DNA hybridization detection using procedures described in EXAMPLE 3. The CNT wrapping strand was chosen to have a segment of the anthrax lethal factor (ALF) GGA TTA TTG TTA AAT ATT GAT AAG GAT in both 5′ and 3′ end. Sandwiched in the middle is a GT repeat (GT)₁₀ as shown in FIG. 4. The DNA-wrapped CNT was prepared as described in Example 1 and was denoted as ALF-OKCNT. Magnetic beads were labeled as described in Example 2 with either cALF (=ATC CTT ATC MT ATT TAA CAA TAA TCC), which is complementary to the ALF sequence, or NZF (=AAG GGT TCA GCG TGG GCG), which is not complementary to the ALF sequence. The resultant beads were denoted as cALF-MB and NZF-MB, respectively. Two hybridization reactions were set up (ALF-OKCNT+cALF-MB, and ALF-OKCNT+NZF-MB). After hybridization, the amount of CNTs remaining in each of the reaction vials were measured by UV-Vis absorption spectrometry at 990 nm where the CNT mixture has a maximum absorption peak. As shown in FIG. 4, removal of CNT by the magnetic beads is hybridization specific.

1. A method of labeling a target nucleic acid molecule, comprising:
a) providing a target nucleic acid molecule having a target sequence;
b) providing a solution containing a population of singly dispersed, carbon nanotube—nucleic acid complexes, each complex comprising a single walled carbon nanotube non-covalently associated with a dispersant nucleic acid molecule wherein the nucleic acid molecule comprises a sequence complementary to the target sequence;
c) hybridizing the target nucleic acid molecule of step (a) to the dispersant nucleic acid molecule of step (b) to form a hybridized complex wherein the target nucleic acid molecule is labeled; and
d) optionally recovering the labeled target nucleic acid molecule.

2. The method according to claim 1 wherein either the target nucleic acid molecule or the dispersant nucleic acid molecule is selected from the group consisting of; single stranded DNA, double stranded DNA, RNA and PNA.

3. A method according to claim 1 wherein the dispersant nucleic acid molecule comprises a sequence selected from the group consisting of:
a) An wherein n=1-2000;
b) Tn wherein n=1-2000;
c) Cn wherein n=1-2000;
d) Gn wherein n=1-2000;
e) Rn wherein n=1-2000, and wherein R may be either A or G;
f) Yn wherein n=1-2000, and wherein Y may be either C or T;
g) Mn wherein n=1-2000, and wherein M may be either A or C;
h) Kn wherein n=1-2000, and wherein K may be either G or T;
i) Sn wherein n=1-2000, and wherein S may be either C or G;
j) Wn wherein n=1-2000, and wherein W may be either A or T;
k) Hn wherein n=1-2000, and wherein H may be either A or C or T;
l) Bn wherein n=1-2000, and wherein B may be either C or G or T;
m) Vn wherein n=1-2000, and wherein V may be either A or C or G;
n) Dn wherein n=1-2000, and wherein D may be either A or G or T; and
o) Nn wherein n=1-2000, and wherein N may be either A or C or T or G.

4. The method according to claim 1 wherein either one or both of the target nucleic acid molecule or the dispersant nucleic acid molecule is functionalized with a member of a binding pair.

5. The method according to claim 4 wherein the member of a binding pair is selected from one of the binding pairs selected from the group consisting of antigen/antibody, hapten/anti-hapten, biotin/avidin, biotin/streptavidin, folic acid/folate binding protein, hormone/hormone receptor, lectin/specific carbohydrate, enzyme/enzyme substrate, enzyme/enzyme inhibitor, and vitamin B12/intrinsic factor.

6. The method of claim 1 wherein the target nucleic acid molecule is immobilized on a solid support.

7. The method of claim 6 wherein the solid support is a magnetic bead.

8. A method for the detection of a target nucleic acid molecule, comprising:
a) providing a target nucleic acid molecule having a target sequence;
b) providing a solution containing a population of singly dispersed, carbon nanotube—nucleic acid complexes, each complex comprising a single walled carbon nanotube non-covalently associated with a dispersant nucleic acid molecule wherein the nucleic acid molecule comprises a sequence complementary to the target sequence;
c) hybridizing the target nucleic acid molecule of step (a) to the dispersant nucleic acid molecule of step (b) to form a hybridized complex; and
d) detecting the hybridization of step (c) by measuring change in properties of the carbon nanotube, before and after hybridization, wherein the target nucleic acid molecule is detected.

9. A method for the detection of a target nucleic acid molecule, comprising:
a) providing a target nucleic acid molecule having a target sequence;
b) providing a solution containing a population of singly dispersed, carbon nanotube—nucleic acid complexes, each complex comprising a single walled carbon nanotube non-covalently associated with a dispersant nucleic acid molecule wherein the nucleic acid molecule comprises a sequence complementary to the target sequence;
c) proving a solid support comprising a linking nucleic acid molecule, further comprising a first hybridization sequence complementary to the target sequence and a second hybridization sequence complementary to at least a portion of the dispersant nucleic acid molecule:
d) hybridizing the target nucleic acid molecule of step (a) and the dispersant nucleic acid molecule of step (b) to the linking nucleic acid molecule of step (c) to form an immobilized complex; and
e) detecting the hybridization of step (d) by measuring changes in properties of the carbon nanotube, before and after hybridization, wherein the target nucleic acid is detected.

10. A method for the detection of a target nucleic acid molecule, comprising:
a) providing a target nucleic acid molecule having a target sequence wherein the target sequence is immobilized on a solid support;
b) providing a solution containing a population of singly dispersed, carbon nanotube—nucleic acid complexes, each complex comprising a single walled carbon nanotube non-covalently associated with a dispersant nucleic acid molecule wherein the nucleic acid molecule comprises a sequence complementary to the target sequence;
c) hybridizing the target nucleic acid molecule of step (a) and the dispersant nucleic acid molecule of step (b) to form an immobilized complex; and
d) detecting the hybridization of step (c) by measuring change in properties of the carbon nanotube, before and after hybridization, wherein the target nucleic acid is detected.

11. The method of any of claims 8, 9 or 10 wherein the change in properties of the carbon nanotube are a change in conductive properties.

12. The method of any of claims 8, 9 or 10 wherein the change in properties of the carbon nanotube are a change in electrochemical properties.

13. The method of any of claims 8, 9 or 10 wherein the change in properties of the carbon nanotube are a change in spectral properties.

14. The method any of claims 8, 9 or 10 wherein the target nucleic acid is an indicator of disease.

15. The method of claim 14 wherein the disease is selected from the group consisting of cancers, adrenal hyperplasia, myotonic dystrophy, hypothyroidism, cataract development, tay-sachs disease, retinoblastoma, blood group antigen mutations, and neurofibromatosis

16. The method of any of claims 8, 9 or 10 wherein the target nucleic acid molecule is isolated from an organism selected from the group consisting of bacteria, yeast, fungi, viruses, plants, and mammals.

17. The method of claims 9 or 10 wherein the solid support is selected from the group consisting of; magnetic beads, glass, films, synthetic polymer supports, agarose, nitrocellulose, and nylon® supports.

18. The method of any of claims 8, 9 or 10 wherein the target nucleic acid is isolated from a biological fluid.

19. The method of claim 18 wherein the biological fluid is selected from the group consisting of plasma, serum, spinal fluid, lymph fluid, synovial fluid, urine, and tears.

20. The method of any of claims 8, 9 or 10 wherein the singly dispersed, nanotube—nucleic acid complexes are comprised within a field-effect transistor.


SEQUENCE LISTING (3 sequences)

SEQ ID NO: 1 - DNA, 27 bp, Synthetic
ggattattgttaaatattgataaggat

SEQ ID NO: 2 - DNA, 27 bp, Synthetic
atccttatcaatatttaacaataatcc

SEQ ID NO: 3 - DNA, 18 bp, Synthetic
aagggttcagcgtgggcg